SEQUENCE LISTING (58 sequences)

SEQ ID NO: 1 - DNA, 1071 bp, Neisseria cinerea
  source 1..1071: mol_type = genomic DNA
atgaaaatcgttttgaccacttctatggcgggtctgggcggcacggaaaacgccaccttccgcctcggccggctgctcaggcagcacggacacgacatcatccttgcctcttcggacggaccgttgctcaaagaggcgcaagccttgggcatacgttggcaacccgtcgatttctatcgcggcggccttttcggctacttcaaaggcatgattgcctacgcccgaatgctgcggcgcgaacaacccgacatcatccactgccaaatggcgcgcatcgtgcccgcctgcgccgttgccgccaaaatcgtttcgccgaaaaccaaagtgttctaccacgcacgcgggctgaaccccgaaacctatccgaaaatcgcccggctgttcgaccgtttgggcgtgtacatcatcggcaactgcaaacacgagcgcgacaaactcatccgctacggttttcccgcacaccgcatcacttatacctacaacgcgctgcaccgtcccgattccgttcccgaaaaaaccgccaaagactgcgtcatgctcggcacgctgtcgcgtctggacactgtccgcgccgtgcatctgatgctggacattttcaaaaaaatggtcgaccgcgatatgcccgtacgcctgaacgtggcgggcataggcgaagaaatggacaatttgaaagcgcaggcggaacgtctgggcatagacggcaaagttaccttcctcggcggcgtgcgcgatttgaccgcctacttcaaagacgtggatattttggtgaacacgccccactgcatcggcgaccacggcgcaggtgtgggcaacaacatcttagaagccggactttacgacacgcccgtcgttacctacgatatggcgggcatttccgaaatggtcgtcaacggggaaacgggttactgcatccccttcggcgacgaagaagcctttatcgccgccgtagacacgctcgtcagacagccggagttgcgcgaagcaatgggcaaaaggctgcacacacacgtcgccgcactctgttccgacgacgaaatctaccgtaccaccatggccgcctataccatgtaa

SEQ ID NO: 2 - DNA, 1089 bp, Neisseria meningitidis
  source 1..1089: mol_type = genomic DNA
atgccgtctgaagccttcagacggcatcgtgcctatcgggagaataaattgcagcctttagtcagcgtattgatttgcgcctacaacgtagaaaaatatttcgcccaatcattagccgccgtcgtgaatcagacttggcgcaacttggatattttgattgtcgatgacggctcgacagacggtacgcttgccattgcccagcgtttccaagaacaggacggtcgtatccgcatcctcgcgcagccacgcaattccggtctgattccctctttaaacatcgggctggacgaattggcaaagtcaggaatgggggaatatattgcacgcaccgatgccgacgatattgccgcccccgactggattgagaaaatcgtgggcgagatggaaaaagaccgcagcatcattgccatgggtgcgtggctggaagttttgtcggaagaaaaggacggcaaccggctggcgcggcatcacaggcacggcaaaatttggaaaaagccgacccgacacgaagatattgccgactttttccctttcggcaaccccatacacaacaacacgatgattatgaggcgcagcgtcattgacggcggattgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgccgcccgccggcgcgtggctggatttcgcggcagacggcaagatgaggcggctgtttaccatgaggcaatacttcggcattttgcaccggctgattaaaaaccgccggcaggcgcggtcggattcggcagggaaagaacaggagatttaa

SEQ ID NO: 3 - DNA, 840 bp, Neisseria cinerea
  source 1..840: mol_type = genomic DNA
atggaaaaccgtattatcagcctagcttccgccaaagagcgcagggcgcacattgccgataccttcggcaggcacgacattccgtttcagtttttcgacgcactgatgccgtctgaagaactggaacaggcgatgggcgagttggttcccggtttggttcggcacaacctgttgagtagtgtggaaaaagcctgtttcatgagccacgtcgtattgtggaagcaggcactggacgaaggcgtatcgtatattgccgtatttgaagatgatgtcttactcggcgaaggggcggagaaattccttgccaaagacggctggctaaaagagcggtttgacccggattccgcctttatcgtccgtttggaaacgatgtttataaaagtcattgctaggccgtgtggggttcctgattatgaaggcagacatttcccattattggaaagtgagcattgggggacggcaggttatattatttcccgtgaggcgatgcggtttttcctgcaacatttcgcaatgttgcagccggaacggataaaacctatcgacctaatgatgttcagtgattttttcgataaggaggggatgcctgtttatcaggtcagtccggccttatgcgcccaagaattgcattacactaagtttcataataaaagcagcgtgttgggtagtttattggaagatgagcgaagtaaaatccaaaaacaaaatatttctcaaacacataaagacaggacactgaaagagcgattgattcatgcattgacaaaaatcagcagggaaaagaaaaaacgggaggaaagaaagaaaatgagtgaggcggttatcataccgttccggtaa

SEQ ID NO: 4 - DNA, 828 bp, Lutiella nitroferrum
  source 1..828: mol_type = genomic DNA
atggataagattaaacaaggatctgccagtctagtggtaggcgatcaacaagaaaaacaccccgttgtttctgttttgcttcctgtaaatagggtagatcggttttttatccctgcagttgagagcattcttacccagacattgcaagattttgaattaatcattatagcaaatggctgtagcaccgagcatttaaacaaaattaggttgacatatggcgaccataatagggtaagaatcctgaacactgaaatcaaaggacttccttttgcgcttaatttaggagtacataacgcacgcggtttatatattgccagaatggatgccgacgacatatccattcccgaaaggctcgagaaacagttgaatacccttgaacagaataaaaaaataggagtggtttcgtcgggggttgattttattgatgaaaacgaccaagcaattagagaaggtaaatttcctgaactaacggacaaagaccatagaagactgcttccattgatctgctgcattgcacatccaactgtgatggttcgaaaggaaattatcaataagttgggtggttattcttttggtagcttctcagaagattatgatttatggcttaggataatgcgcgagctgcccgaggtagaattctaccgcataccggaaagcctattgaaatatagaagacatggaaatcaagcaacctcatcaaaaaatataaagaaaattagggcgtataactccgcactaaagataagagagttatttttaagcaggaaactaaagtttattattggtatcattctccctgccagaatggtgacgctatggagaaagtaa

SEQ ID NO: 5 - DNA, 1254 bp, Escherichia coli
  source 1..1254: mol_type = genomic DNA
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

SEQ ID NO: 6 - DNA, 53 bp, synthetic construct
  source 1..53: mol_type = other DNA
tccaggaggacatacaaccgagaaggagggttattagatgccgtctgaagcct

SEQ ID NO: 7 - DNA, 40 bp, synthetic construct
  source 1..40: mol_type = other DNA
cctttatgcgcaacgttaaatctcctgttctttccctgcc

SEQ ID NO: 8 - DNA, 58 bp, synthetic construct
  source 1..58: mol_type = other DNA
aacaggagatttaacgttgcgcataaaggagcatctacaatgtactatttaaaaaaca

SEQ ID NO: 9 - DNA, 46 bp, synthetic construct
  source 1..46: mol_type = other DNA
ttgtcgacggagctcgaattctttaagcgacttcattcacctgacg

SEQ ID NO: 10 - DNA, 62 bp, synthetic construct
  source 1..62: mol_type = other DNA
tccaggaggacatacaaccgagaaggagggttattagtctagagatgcagcccctagtcagc

SEQ ID NO: 11 - DNA, 42 bp, synthetic construct
  source 1..42: mol_type = other DNA
cattaataatcctccttctgtcaacggtttttcaacaaccgg

SEQ ID NO: 12 - DNA, 40 bp, synthetic construct
  source 1..40: mol_type = other DNA
tgacagaaggaggattattaatggaaaaccgtattatcag

SEQ ID NO: 13 - DNA, 46 bp, synthetic construct
  source 1..46: mol_type = other DNA
atgctcctttatgcgcaacgccgcggttaccggaacggtatgataa

SEQ ID NO: 14 - DNA, 80 bp, synthetic construct
  source 1..80: mol_type = other DNA
ttatcataccgttccggtaaccgcggcgttgcgcataaaggagcatctacaatgtactatttaaaaaacacaaacttttg

SEQ ID NO: 15 - DNA, 40 bp, synthetic construct
  source 1..40: mol_type = other DNA
aagcttgtcgacggagctcgttaagcgacttcattcacct

SEQ ID NO: 16 - DNA, 53 bp, synthetic construct
  source 1..53: mol_type = other DNA
gcaaactatgatgggtcttgttgttggattctagataacgtgggcgatcgatg

SEQ ID NO: 17 - DNA, 51 bp, synthetic construct
  source 1..51: mol_type = other DNA
ggggctgcatctaataaccctccttctgatatcgccgtactccttggagat

SEQ ID NO: 18 - DNA, 37 bp, synthetic construct
  source 1..37: mol_type = other DNA
atcagaaggagggttattagatgcagcccctagtcag

SEQ ID NO: 19 - DNA, 47 bp, synthetic construct
  source 1..47: mol_type = other DNA
atgctcctttccgaaactccgtatactcaacggtttttcaacaaccg

SEQ ID NO: 20 - DNA, 46 bp, synthetic construct
  source 1..46: mol_type = other DNA
ttcggaaaggagcatctaggatggataagattaaacaaggatctgc

SEQ ID NO: 21 - DNA, 42 bp, synthetic construct
  source 1..42: mol_type = other DNA
ctttatgcgcaacgggatccttactttctccatagcgtcacc

SEQ ID NO: 22 - DNA, 45 bp, synthetic construct
  source 1..45: mol_type = other DNA
cgttgcgcataaaggagcatctacaatgtactatttaaaaaacac

SEQ ID NO: 23 - DNA, 40 bp, synthetic construct
  source 1..40: mol_type = other DNA
tgcatgcctgcaggtcgacttcacgagcccctcattgcct

SEQ ID NO: 24 - DNA, 40 bp, synthetic construct
  source 1..40: mol_type = other DNA
cattcgcagggtaacggccagactttacaacaactttttc

SEQ ID NO: 25 - DNA, 40 bp, synthetic construct
  source 1..40: mol_type = other DNA
gaaaaagttgttgtaaagtctggccgttaccctgcgaatg

SEQ ID NO: 26 - DNA, 40 bp, synthetic construct
  source 1..40: mol_type = other DNA
acaattccacacatgcgcattgtatgtcctcctggacttc

SEQ ID NO: 27 - DNA, 40 bp, synthetic construct
  source 1..40: mol_type = other DNA
gaagtccaggaggacatacaatgcgcatgtgtggaattgt

SEQ ID NO: 28 - DNA, 40 bp, synthetic construct
  source 1..40: mol_type = other DNA
gctcggtacccggggatcctaaagcaccctcaaggcgctg

SEQ ID NO: 29 - DNA, 36 bp, synthetic construct
  source 1..36: mol_type = other DNA
ctatgaccatgattacgccactccggcgagttcaag

SEQ ID NO: 30 - DNA, 39 bp, synthetic construct
  source 1..39: mol_type = other DNA
cattcgcagggtaacggccagcgattaattatgcacggc

SEQ ID NO: 31 - DNA, 36 bp, synthetic construct
  source 1..36: mol_type = other DNA
aggccgtgcataattaatcgctggccgttaccctgc

SEQ ID NO: 32 - DNA, 39 bp, synthetic construct
  source 1..39: mol_type = other DNA
gttccaaatagtcgagtcattgtatgtcctcctggactt

SEQ ID NO: 33 - DNA, 42 bp, synthetic construct
  source 1..42: mol_type = other DNA
gaagtccaggaggacatacaatgactcgactatttggaactg

SEQ ID NO: 34 - DNA, 41 bp, synthetic construct
  source 1..41: mol_type = other DNA
ttgtaaaacgacggccagtgttcaggtgctctaggtaacgg

SEQ ID NO: 35 - DNA, 40 bp, synthetic construct
  source 1..40: mol_type = other DNA
tgcatgcctgcaggtcgactctctggaatctggtcggatc

SEQ ID NO: 36 - DNA, 40 bp, synthetic construct
  source 1..40: mol_type = other DNA
cattcgcagggtaacggccagattatctcaaatccttaaa

SEQ ID NO: 37 - DNA, 40 bp, synthetic construct
  source 1..40: mol_type = other DNA
tttaaggatttgagataatctggccgttaccctgcgaatg

SEQ ID NO: 38 - DNA, 40 bp, synthetic construct
  source 1..40: mol_type = other DNA
gagaaatcgcttgcgctcaatgtatgtcctcctggacttc

SEQ ID NO: 39 - DNA, 40 bp, synthetic construct
  source 1..40: mol_type = other DNA
gaagtccaggaggacatacattgagcgcaagcgatttctc

SEQ ID NO: 40 - DNA, 40 bp, synthetic construct
  source 1..40: mol_type = other DNA
gctcggtacccggggatccttgctcaacgatggcggtgac

SEQ ID NO: 41 - DNA, 40 bp, synthetic construct
  source 1..40: mol_type = other DNA
tgcatgcctgcaggtcgactacacgccagggtattcgccg

SEQ ID NO: 42 - DNA, 40 bp, synthetic construct
  source 1..40: mol_type = other DNA
cattcgcagggtaacggccagtttgctccttaaaacacca

SEQ ID NO: 43 - DNA, 40 bp, synthetic construct
  source 1..40: mol_type = other DNA
tggtgttttaaggagcaaactggccgttaccctgcgaatg

SEQ ID NO: 44 - DNA, 40 bp, synthetic construct
  source 1..40: mol_type = other DNA
ccggcgcgttcatgtgccattgtatgtcctcctggacttc

SEQ ID NO: 45 - DNA, 40 bp, synthetic construct
  source 1..40: mol_type = other DNA
gaagtccaggaggacatacaatggcacatgaacgcgccgg

SEQ ID NO: 46 - DNA, 40 bp, synthetic construct
  source 1..40: mol_type = other DNA
gctcggtacccggggatcctttgtatttgaatccgccatc

SEQ ID NO: 47 - DNA, 40 bp, synthetic construct
  source 1..40: mol_type = other DNA
tgcatgcctgcaggtcgacttcgtagaaaccgccaccttt

SEQ ID NO: 48 - DNA, 40 bp, synthetic construct
  source 1..40: mol_type = other DNA
cattcgcagggtaacggccaggaaccaagagtacctgccc

SEQ ID NO: 49 - DNA, 40 bp, synthetic construct
  source 1..40: mol_type = other DNA
gggcaggtactcttggttcctggccgttaccctgcgaatg

SEQ ID NO: 50 - DNA, 40 bp, synthetic construct
  source 1..40: mol_type = other DNA
tcatcgataggcaaactcattgtatgtcctcctggacttc

SEQ ID NO: 51 - DNA, 40 bp, synthetic construct
  source 1..40: mol_type = other DNA
gaagtccaggaggacatacaatgagtttgcctatcgatga

SEQ ID NO: 52 - DNA, 38 bp, synthetic construct
  source 1..38: mol_type = other DNA
gctcggtacccggggatcctcaaaggacagatccaccg

SEQ ID NO: 53 - DNA, 40 bp, synthetic construct
  source 1..40: mol_type = other DNA
tgcatgcctgcaggtcgactctccagagggacgttccctc

SEQ ID NO: 54 - DNA, 40 bp, synthetic construct
  source 1..40: mol_type = other DNA
cattcgcagggtaacggccacgtgtgttagccctcaacct

SEQ ID NO: 55 - DNA, 40 bp, synthetic construct
  source 1..40: mol_type = other DNA
aggttgagggctaacacacgtggccgttaccctgcgaatg

SEQ ID NO: 56 - DNA, 40 bp, synthetic construct
  source 1..40: mol_type = other DNA
ccggtaaccagaagcttcattgtatgtcctcctggacttc

SEQ ID NO: 57 - DNA, 40 bp, synthetic construct
  source 1..40: mol_type = other DNA
gaagtccaggaggacatacaatgaagcttctggttaccgg

SEQ ID NO: 58 - DNA, 40 bp, synthetic construct
  source 1..40: mol_type = other DNA
gctcggtacccggggatcctaagtagcgcaagctggttgc

(19) United States
(12) Patent Application Publication
(10) Pub. No.: US 2025/0313874 A1
(43) Pub. Date: Oct. 9, 2025

(54) METHOD FOR PRODUCING LACTO-N-TETRAOSE AND LACTO-N-NEOTETRAOSE USING CORYNEBACTERIUM GLUTAMICUM

(71) Applicant: ADVANCED PROTEIN TECHNOLOGIES CORP., Suwon-si, Gyeonggi-do (KR)

(72) Inventors: Chul Soo SHIN, Suwon-si (KR); Jong Won YOON, Seongnam-si (KR); Young Ha SONG, Yongin-si (KR); Young Sun YOU, Yongin-si (KR); Su Jin KANG, Hwaseong-si (KR); Chang Yun CHOI, Osan-si (KR)

(73) Assignee: ADVANCED PROTEIN TECHNOLOGIES CORP., Suwon-si, Gyeonggi-do (KR)

(21) Appl. No.: 18/864,765

(22) PCT Filed: May 11, 2023
(86) PCT No.: PCT/KR2023/006403

§ 371 Date: Nov. 11, 2024

(30) Foreign Application Priority Data
May 11, 2022 (KR) 10-2022-0057992
May 10, 2023 (KR) 10-2023-0060393

(51) Int. Cl. C12P 19/02; C12N 9/10; C12N 9/12; C12N 9/90

(52) U.S. Cl. CPC C12P 19/02; C12N 9/1029; C12N 9/1051; C12N 9/1096; C12N 9/1205; C12N 9/1241; C12N 9/90; C12Y 203/01157; C12Y 206/01016; C12Y 207/07009; C12Y 501/03002; C12Y 504/02002; C12Y 504/0201

(57) ABSTRACT

The present invention relates to a method for producing lacto-N-tetraose (LNT) and lacto-N-neotetraose (LNnT) using Corynebacterium glutamicum, and more specifically to: recombinant Corynebacterium glutamicum transformed such that, in order to increase productivity of LNT and LNnT, genes introduced from outside are expressed in Corynebacterium glutamicum, and genes inherent in Corynebacterium glutamicum are overexpressed; and a method for producing LNT and LNnT using same. Accordingly, the present invention uses Corynebacterium glutamicum so as to enable producing LNT and LNnT in a safe manner and in high concentration, high yield, high productivity, compared to when using conventional Escherichia coli.

### TECHNICAL FIELD

The present invention relates to a method of producing lacto-N-tetraose (LNT) and lacto-N-neotetraose (LNnT) using Corynebacterium glutamicum, and more specifically to recombinant Corynebacterium glutamicum transformed such that exogenous genes are expressed in Corynebacterium glutamicum, and genes inherent in Corynebacterium glutamicum are overexpressed, in order to increase productivity of LNT and LNnT, and a method of producing LNT and LNnT using the same.

### BACKGROUND ART

Human milk oligosaccharides (HMOS) are oligosaccharides contained in human milk and are the third most abundant component after lactose and fat. There are about 200 types of various human milk oligosaccharides. Representative examples of human milk oligosaccharides include 2′-fucosyllactose (2′-FL), 3-fucosyllactose (3-FL), lacto-N-triose II, lacto-N-tetraose (LNT), lacto-N-neotetraose (LNnT), lacto-N-fucopentaose (LNFP), lacto-N-neofucopentaose, lacto-N-hexaose (LNH), lacto-N-neohexaose (LNnH), 6′-galactosylactose, 3′-galactosylactose and the like.

Human milk oligosaccharides have advantages of strengthening the immune function or having positive effects on the development and behaviors of children. Therefore, there is a need for continued research on technologies for producing various human milk oligosaccharides. In previous studies, research has been conducted on methods for producing human milk oligosaccharides using microorganisms, in particular, E. coli. However, E. coli is recognized as a harmful germ by consumers and E. coli cells are limitedly used due to a phenomenon called “lactose killing” in which E. coli cells are killed under lactose-restricted culture by lactose permease. Accordingly, there is a continuing need for technology to produce human milk oligosaccharides using novel microorganisms.

### DISCLOSURE

### Technical Problem

Therefore, it is an object of the present invention to develop and provide a method for producing lacto-N-tetraose (LNT) and lacto-N-neotetraose (LNnT) at a high concentration, high yield and high productivity using Corynebacterium glutamicum, which is safer than Escherichia coli, as a host cell for producing the LNT and LNnT, which are food and pharmaceutical substances.

### Technical Solution

In accordance with one aspect of the present invention, provided is a recombinant Corynebacterium glutamicum transformed such that exogenous genes, including genes encoding lactose permease, genes encoding β-1, 3-N-acetylglucosaminyltransferase, and genes encoding β-1,3-galactosyltransferase are expressed in Corynebacterium glutamicum, the recombinant Corynebacterium glutamicum transformed such that one or more genes selected from endogenous genes in Corynebacterium glutamicum, including genes encoding glutamine-fructose-6-phosphate aminotransferase, genes encoding phosphoglucosamine mutase, genes encoding glucosamine-1-phosphate N-acetyltransferase, genes encoding UDP-N-acetylglucosamine pyrophosphorylase, genes encoding phosphoglucomutase, genes encoding UTP-glucose-1-phosphate uridylyltransferase, and genes encoding UDP-glucose-4-epimerase are overexpressed.

In accordance with another aspect of the present invention, provided is a recombinant Corynebacterium glutamicum transformed such that exogenous genes, including genes encoding lactose permease, genes encoding β-1,β-N-acetylglucosaminyltransferase, and genes encoding β-1, 4-galactosyltransferase, are expressed in Corynebacterium glutamicum, the recombinant Corynebacterium glutamicum transformed such that one or more genes selected from endogenous genes in Corynebacterium glutamicum, including genes encoding glutamine-fructose-6-phosphate aminotransferase, genes encoding phosphoglucosamine mutase, genes encoding glucosamine-1-phosphate N-acetyltransferase, genes encoding UDP-N-acetylglucosamine pyrophosphorylase, genes encoding phosphoglucomutase, genes encoding UTP-glucose-1-phosphate uridylyltransferase, and genes encoding UDP-glucose-4-epimerase, are overexpressed.

In accordance with another aspect of the present invention, provided is a method for producing lacto-N-tetraose comprising culturing the recombinant Corynebacterium glutamicum according to claim 1 in a medium containing lactose.

Preferably, the medium further contains glucose.

In accordance with another aspect of the present invention, provided is a method for producing lacto-N-neotetraose comprising culturing the recombinant Corynebacterium glutamicum according to claim 2 in a medium containing lactose.

Preferably, the medium may further contain glucose.

### Advantageous Effects

The present invention enables production of lacto-N-tetraose (LNT) and lacto-N-neotetraose (LNnT) using Corynebacterium glutamicum at a high concentration, a high yield, and high productivity, in a safer manner than conventional E. coli.

### DESCRIPTION OF DRAWINGS

FIG. 1 is a flowchart illustrating a pathway for

biosynthesizing lacto-N-tetraose (LNT) in a recombinant Corynebacterium glutamicum strain of the present invention.

FIG. 2 is a flowchart illustrating a pathway for biosynthesizing lacto-N-neotetraose (LNnT) in a recombinant Corynebacterium glutamicum strain of the present invention.

FIG. 3 is a graph showing comparison in the production amount of lacto-N-trioseII (LNTII) of the recombinant Corynebacterium glutamicum strain produced to overexpress glms, glmM, and glmU of the production pathway of UDP-N-acetylglucosamine, a precursor substance in the present invention.

FIG. 4 is a graph showing comparison in the production amount (final production amount) of LNT/LNnT of the recombinant Corynebacterium glutamicum strain produced to overexpress pgm, galU, and galE of the production pathway of UDP-galactose, a precursor substance in the present invention.

FIG. 5 is a graph showing the LNT/LNnT production amount over time of the recombinant Corynebacterium glutamicum strain produced to overexpress pgm, galU, and galE of the production pathway of UDP-galactose, a precursor substance in the present invention.

### BEST MODE

Methods for producing various human milk oligosaccharides have been continuously researched because human milk oligosaccharides have advantages of strengthening the immune function or having positive effects on the development and behaviors of children. Previous studies have been conducted on methods for producing human milk oligosaccharides using microorganisms and there is an increasing need to produce various human milk oligosaccharides using novel microorganisms.

Here, Corynebacterium glutamicum was used as the host cell for the production of lacto-N-neotetraose (LNnT) and lacto-N-tetraose (LNT). Unlike conventionally used Escherichia coli, Corynebacterium glutamicum is considered to be a GRAS (generally recognized as safe) strain which is widely used for industrially producing amino acids and nucleic acids as food additives. In addition, there is a strong perception that E. coli is a harmful bacterium to consumers, and there is a limitation in that it costs a lot to isolate and purify the produced human milk oligosaccharides because the cell membrane components of E. coli may act as endotoxins. However, E. coli cells are limitedly used due to a phenomenon called “lactose killing” in which E. coli cells are killed under lactose-restricted culture by lactose permease. Accordingly, Corynebacterium glutamicum is considered to be a safe strain suitable for the production of food and pharmaceutical materials.

Accordingly, the present invention provides recombinant Corynebacterium glutamicum transformed such that exogenous genes, namely, genes encoding lactose permease, genes encoding β-1, 3-N-acetylglucosaminyltransferase, and genes encoding β-1, 3-galactosyltransferase are expressed in Corynebacterium glutamicum, and transformed such that one or more genes selected from endogenous genes in Corynebacterium glutamicum, namely, genes encoding glutamine-fructose-6-phosphate aminotransferase, genes encoding phosphoglucosamine mutase, genes encoding glucosamine-1-phosphate N-acetyltransferase, genes encoding UDP-N-acetylglucosamine pyrophosphorylase, genes encoding phosphoglucomutase, genes encoding UTP-glucose-1-phosphate uridylyltransferase, and genes encoding UDP-glucose-4-epimerase are overexpressed. In addition, the present invention provides a method of producing lacto-N-tetraose including culturing the recombinant Corynebacterium glutamicum in a medium containing lactose.

Accordingly, the present invention provides recombinant Corynebacterium glutamicum transformed such that exogenous genes, namely, genes encoding lactose permease, genes encoding β-1, 3-N-acetylglucosaminyltransferase, and genes encoding β-1, 4-galactosyltransferase are expressed in Corynebacterium glutamicum and one or more genes selected from endogenous genes in Corynebacterium glutamicum, namely, genes encoding glutamine-fructose-6-phosphate aminotransferase, genes encoding phosphoglucosamine mutase, genes encoding glucosamine-1-phosphate N-acetyltransferase, genes encoding UDP-N-acetylglucosamine pyrophosphorylase, genes encoding phosphoglucomutase, genes encoding UTP-glucose-1-phosphate uridylyltransferase, and genes encoding UDP-glucose-4-epimerase are overexpressed. In addition, the present invention provides a method of producing lacto-N-neotetraose including culturing the recombinant

Corynebacterium glutamicum in a medium containing lactose. The process for producing LNT and LNnT using the

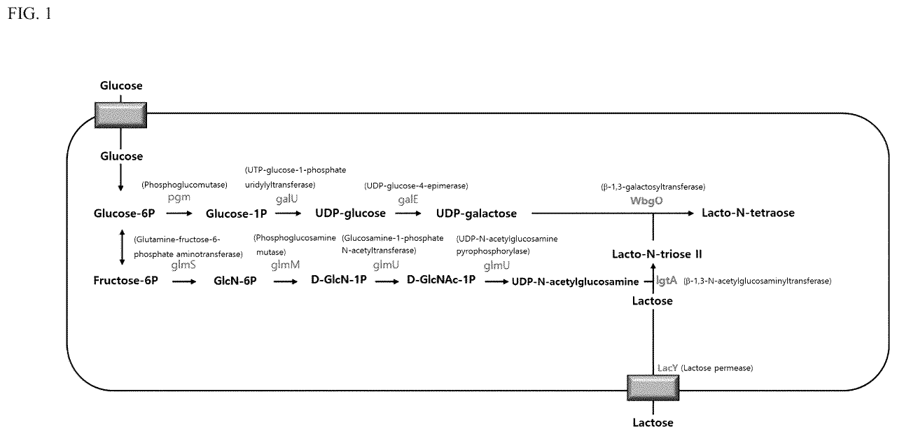

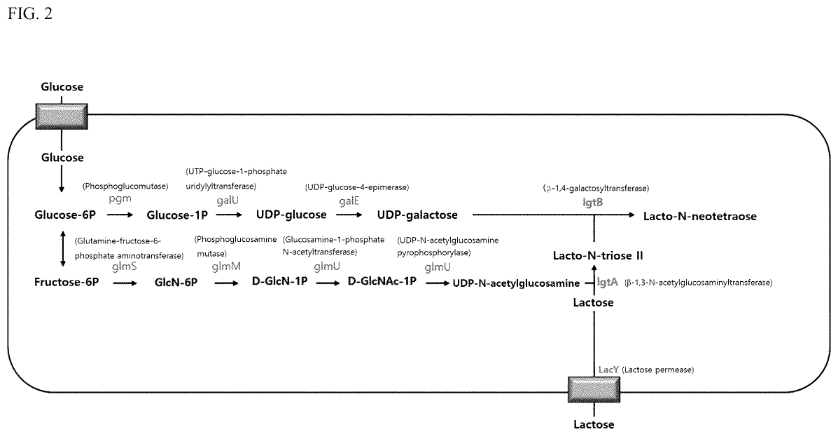

recombinant Corynebacterium glutamicum of the present invention is shown in FIGS. 1 and 2. When lactose reacts with UDP-N-acetylglucosamine (UDP-N-GlcNAc), which is one of the precursor substances, β-1, 3-N-acetylglucosaminyltransferase (encoded by lgtA) catalyzes production of lacto-N-trioseII (LNTII). The produced LNTII reacts with another precursor substance, UDP-galactose. At this time, β-1, 3-galactosyltransferase (encoded by WbgO) catalyzes production of LNT (FIG. 1), or β-1, 4-galactosyltransferase (encoded by lgtB) catalyzes production of LNnT (FIG. 2).

Meanwhile, the recombinant Corynebacterium glutamicum of the present invention is transformed such that a gene encoding lactose permease is expressed, and the lactose permease is an enzyme involved in transporting lactose present outside the strain into the strain and is preferably derived from E. coli, for example, LacY.

Meanwhile, the recombinant Corynebacterium glutamicum of the present invention is transformed such that a gene encoding beta-1, 3-N-acetylglucosaminyltransferase (lgtA) is expressed, and the gene encoding beta-1, 3-N-acetylglucosaminyltransferase is derived from, for example, Neisseria meningitidis or Neisseria cinerea, more preferably, Neisseria meningitidis M98 or Neisseria cinerea ATCC 14685.

Meanwhile, the recombinant Corynebacterium glutamicum of the present invention is transformed such that a gene encoding β-1, 3-N-acetylglucosaminyltransferase for LNT production is expressed, and the gene encoding β-1, 3-N-acetylglucosaminyltransferase is, for example, lgtA, and preferably, is derived from Neisseria cinerea. In addition, the recombinant Corynebacterium glutamicum is transformed such that a gene encoding β-1, 3-galactosyltransferase is expressed, and the gene encoding β-1, 3-galactosyltransferase is, for example, WbgO, and preferably WbgO derived from Lutiella nitroferrum, more preferably, WbgO derived from Lutiella nitroferrum ATCC BAA-1479.

In addition, the recombinant Corynebacterium glutamicum of the present invention is transformed such that a gene encoding β-1, 3-N-acetylglucosaminyltransferase for LNnT production is expressed, and the gene encoding β-1, 3-N-acetylglucosaminyltransferase is, for example, lgtA, preferably lgtA derived from Neisseria meningitidis. In addition, the recombinant Corynebacterium glutamicum of the present invention is transformed such that a gene encoding β-1, 4-galactosyltransferase is expressed, and the gene encoding β-1, 4-galactosyltransferase is, for example, lgtB, preferably lgtB derived from Neisseria cinerea.

Meanwhile, the recombinant Corynebacterium glutamicum of the present invention is preferably transformed to overexpress one or more genes selected from genes encoding glutamine-fructose-6-phosphate aminotransferase, genes encoding phosphoglucosamine mutase, genes encoding glucosamine-1-phosphate N-acetyltransferase, genes encoding UDP-N-acetylglucosamine pyrophosphorylase, genes encoding phosphoglucomutase, and genes encoding UTP-glucose-1-phosphate uridylyltransferase, which are endogenous genes in Corynebacterium.

In this case, the gene encoding the glutamine-fructose-6-phosphate aminotransferase is preferably glmS and the gene encoding the phosphoglucosamine mutase is preferably glmM. In addition, the gene encoding glucosamine-1-phosphate N-acetyltransferase and the gene encoding UDP-N-acetylglucosamine pyrophosphorylase are preferably glmU.

In this case, the glmU is a gene encoding a bifunctional enzyme having both UDP-N-acetylglucosamine pyrophosphorylase activity and glucosamine-1-phosphate N-acetyltransferase activity (see FIGS. 1 and 2). In addition, the gene encoding phosphoglucomutase is preferably pgm, the gene encoding UTP-glucose-1-phosphate uridylyltransferase is preferably galU, and the gene encoding UDP-glucose-4-epimerase is preferably galE. As such, by overexpressing the genes inherent in Corynebacterium glutamicum, large amounts of UDP-N-acetylglucosamine (UDP-N-GlcNAc) and Lacto-N-triose II (LNTII), which are precursors of LNT and LNnT, are produced and thus the productivity of LNT and LNnT are increased.

Meanwhile, the term “expression” as used herein means incorporation and expression of external genes into strains in order to intentionally express enzymes that cannot be inherently expressed by the Corynebacterium glutamicum strain according to the present invention, and the term “overexpression” as used herein means overexpression that is induced by artificially increasing the amount of expressed enzyme in order to increase expression for mass-production, although the Corynebacterium glutamicum strain according to the present invention has genes encoding the corresponding enzyme and therefore can self-express the same.

Meanwhile, regarding the method for producing lacto-N-tetraose or lacto-N-neotetraose according to the present invention, the medium preferably further contains glucose. By adding glucose to the medium, the growth of a strain can be facilitated, and lacto-N-tetraose or lacto-N-neotetraose can thus be produced at higher productivity.

Meanwhile, according to the following experiment, the recombinant Corynebacterium glutamicum of the present invention was produced to overexpress glms, glmM, and glmU in the production pathway of UDP-N-acetylglucosamine (UDP-N-GlcNAc), a precursor substance, thereby remarkably increasing the production of LNTII, a precursor of LNT/LNnT, and was produced to overexpress pgm, galU, and galE in the production pathway of UDP-galactose, another precursor substance, thereby remarkably increasing the production of LNT/LNnT. As such, the recombinant Corynebacterium glutamicum of the present invention may be used to produce lacto-N-tetraose (LNT) and lacto-N-neotetraose (LNnT) at a high concentration, high yield, and high productivity, in a safer manner than conventional E. coli.

Hereinafter, the present invention will be

described in more detail with reference to the following examples, but the scope of the present invention is not limited to the examples, and includes variations and technical concepts equivalent thereto.

### Example 1: Production of Recombinant Corynebacterium glutamicum and Plasmid

### 1. Construction of Strains for Producing LNT and LNnT

Escherichia coli TOP10 and Corynebacterium glutamicum ATCC 13032 were used, respectively, to construct plasmids and produce lacto-N-triose II (LNTII), lacto-N-tetraose (LNT), and lacto-N-neotetraose (LNnT).
(1) Construction of pAY, Plasmid for LNTII Production (Construction of Plasmid for lgtA-lacY Expression)

The gene (lgtA) encoding β-1, 3-N-acetylglucosaminyltransferase was amplified from Neisseria meningitidis M98 through PCR reaction using two DNA primers 21RBS-lgtA F, lgtA R. In addition, the lacY gene was amplified through PCR reaction using two DNA primers RBS-lacY F and LacY R from the genomic DNA of E. coli K-12 MG1655, and the lgtA-lacY DNA fragment was synthesized through overlap PCR reaction using two DNA primers 21RBS-lgtA F and LacY R, and then was inserted into plasmid pCN013 treated with restriction enzyme EcoRI to construct the pAY plasmid.
(2) Construction of pABY, Plasmid for LNnT Production (Construction of Plasmid for lgtA-lgtB-lacY Expression)

The gene (lgtA) encoding β-1, 3-N-acetylglucosaminyltransferase was amplified from Neisseria meningitidis M98 through PCR reaction using two DNA primers, lgtA_tF and lgtA 20B R. The gene (lgtB) encoding β-1, 4-galactosyltransferase was amplified from Neisseria cinerea ATCC 14685 through PCR reaction using two DNA primers, 20_B1 F and 15_B1 R, and then the lgtA-lgtB DNA fragment was synthesized by overlap PCR reaction using two DNA primers, lgtA_t F and 15_B1 R. Then, the lacY gene was amplified through PCR reaction using two DNA primers, lacY_B F and 20ABY R3 from the genomic DNA of E. coli K-12 MG1655, and the lgtA-lgtB-lacY DNA fragment was synthesized through PCR reaction using two DNA primers, lgtA_t F and 20ABY R3, and then was inserted into plasmid pCN013 treated with restriction enzyme EcoRI to construct the pABY plasmid.
(3) Construction of pAWY, Plasmid for LNT Production (Construction of Plasmid for lgtA-WbgO-lacY Expression)

The pgk promoter was amplified from Corynebacterium glutamicum ATCC 13032 through PCR reaction using two DNA primers pgk F and pgk R. The gene encoding β-N-acetylglucosaminyl transferase (lgtA, or NclgtA; wherein Nc means that lgtA is derived from Neisseria cinerea) was amplified from Neisseria cinerea ATCC 14685 by PCR using two DNA primers, 21NcA F and NcA R, and the gene encoding β-1, 3-galactosyltransferase (WbgO, or LnWbgO; In means that WbgO is derived from Lutiella nitroferrum) was amplified from Lutiella nitroferrum ATCC BAA-1479 by PCR using two DNA primers, LnW F and LnW R. The lacY gene was amplified from the genomic DNA of Escherichia coli K-12 MG1655 through PCR reaction using two DNA primers, 20ABY F3 and 20ABY R3. Then, the pgk-lgtA-WbgO-lacY (i.e., pgk-NclgtA-LnWbgO-lacY; wherein Nc means that lgtA is derived from Neisseria cinerea,and In means that WbgO is derived from Lutiella nitroferrum) DNA fragment was synthesized through overlap PCR reaction using two DNA primers, pgk F and 20ABY R3. The DNA fragment was then inserted into the pCN013 plasmid treated with restriction enzymes EcoRI and EcoRV to construct the pAWY plasmid.

### 2. Construction of Strains Overproducing UDP-N-acetylglucosamine (UDP-N-GlcNAc), Precursor of LNT and LNnT

In order to construct strains for producing LNT and LNnT, strains for overproducing UDP-N-acetylglucosamine (UDP-N-GlcNAc) as a precursor substance were constructed. To this end, as shown in FIGS. 1 and 2, three integration plasmids, pK19mobsacB-tuf-glmS, pK19mobsacB-tuf-glmM, and pK19mobsacB-tuf-glmU, were constructed to overexpress glms, glmM, and glmU in the biosynthetic pathway.
(1) Construction of pK19mobsacB-tuf-glmS Plasmid (Construction of Plasmid for glmS Overexpression)

Three genes were amplified through PCR reaction using three pairs of primers (glmS F1, glmS R1) (glmS F2, glmS R2) (glmS F3, glmS R3) from the genomic DNA of Corynebacterium glutamicum, and then DNA fragments were synthesized using two DNA primers, namely, glms F1 and glmS R3, through overlap PCR reaction and then inserted into XbaI-treated plasmid pK19mobsacB to construct pK19mobsacB-tuf-glmS plasmid.
(2) Construction of pK19mobsacB-tuf-glmM Plasmid (Construction of Plasmid for glmM Overexpression)

Three genes were amplified using three pairs of primers (glmM F1, glmM R1) (glmM F2, glmM R2) (glmM F3, glmM R3) from the genomic DNA of Corynebacterium glutamicum, and then DNA fragments were synthesized through overlap PCR reaction using two DNA primers, namely, glmM F1 and glmM R3,and then were inserted into the plasmid pK19mobsacB treated with HindIII and EcoRI to construct the pK19mobsacB-tuf-glmM plasmid.
(3) Construction of pK19mobsacB-tuf-glmU Plasmid (Construction of Plasmid for glmU Overexpression)

Three genes were amplified using three pairs of primers (glmU F1, glmU R1) (glmU F2, glmU R2) (glmU F3, glmU R3) from the genomic DNA of Corynebacterium glutamicum, and then DNA fragments were synthesized using two DNA primers, namely, glmU F1 and glmU R3, through overlap PCR reaction, and were then inserted into the plasmid pK19mobsacB treated with XbaI to construct the pK19mobsacB-tuf-glmU plasmid.

### 3. Construction of Strain for Overproducing UDP-galactose, Precursor of LNT and LNnT

A strain for overproducing UDP-galactose, another precursor for the biosynthesis of LNT and LNnT, was constructed. For this purpose, three integration plasmids, namely, pK19mobsacB-tuf-pgm, pK19mobsacB-tuf-galU1, and pk19mobsacB-tuf-galE, were constructed to overexpress pgm, galU1, and galE in the biosynthetic pathway, as shown in FIGS. 1 and 2.
(1) Construction of pK19mobsacB-tuf-pgm plasmid (construction of plasmid for pgm overexpression)

Three genes were amplified through PCR reaction using six DNA primers (pgm F1, pgm R1), (pgm F2, pgm R2), and (pgm F3, pgm R4) from the genomic DNA of Corynebacterium glutamicum, and then DNA fragments were synthesized using two DNA primers, namely, pgm F1 and pgm R4 through overlap PCR reaction, and were then inserted into the plasmid pK19mobsacB treated with Xba I to construct the pK19mobsacB-tuf-pgm plasmid.
(2) Construction of pK19mobsacB-tuf-galU1 Plasmid (Construction of Plasmid for galU Overexpression)

Three genes were amplified through PCR reaction using six DNA primers (galU1 F1, galU1 R1), (galU1 F2, galU1 R2), (galU1 F3, galU1 R3) from the genomic DNA of Corynebacterium glutamicum, and then DNA fragments were synthesized using two DNA primers, namely, galU1 F1 and galU1 R3 through overlap PCR reaction, and were then inserted into the plasmid pK19mobsacB treated with XbaI to construct the pK19mobsacB-tuf-galU1 plasmid.
(3) Construction of pK19mobsacB-tuf-galE Plasmid (Construction of Plasmid for galE Overexpression)

Three genes were amplified through PCR reaction using six DNA primers (galE F1, galE R1) (galE F2, galE R2), (galE F3, galE R3) from the genomic DNA of Corynebacterium glutamicum, and then DNA fragments were synthesized through overlap PCR reaction using two DNA primers galE F1 and galE R3, and then inserted into plasmid pK19mobsacB treated with XbaI to construct pK19mobsacB-tuf-galE plasmid.

Meanwhile, the primers, strains, plasmids, and gene sequences used in this example are shown in Tables 1 to 5 below.

TABLE 1
Primers

| Primer names | Sequence (5′→3′) |
|---|---|
| 21RBS-lgtA F | TCCAGGAGGACATACAACCGAGAAGGAGGGTTATTAGATGCCGTCTGA |
| | AGCCT |
| lgtA R | CCTTTATGCGCAACGTTAAATCTCCTGTTCTTTCCCTGCC |
| RBS-lacY F | AACAGGAGATTTAACGTTGCGCATAAAGGAGCATCTACAATGTACTAT |
| | TTAAAAAACA |
| LacY R | TTGTCGACGGAGCTCGAATTCTTTAAGCGACTTCATTCACCTGACG |
| lgtA_t F | TCCAGGAGGACATACAACCGAGAAGGAGGGTTATTAGtctagaGATGC |
| | AGCCCCTAGTCAGC |
| lgtA_20B R | CATTAATAATCCTCCTTCTGTCAACGGTTTTTCAACAACCGG |
| 20_B1 F | TGACAGAAGGAGGATTATTAATGGAAAACCGTATTATCAG |
| 15_B1 R | ATGCTCCTTTATGCGCAACGCCGCGGTTACCGGAACGGTATGATAA |
| lacY_B F | TTATCATACCGTTCCGGTAACCGCGGCGTTGCGCATAAAGGAGCATCT |
| | ACAATGTACTATTTAAAAAACACAAACTTTTG |
| 20ABY R3 | AAGCTTGTCGACGGAGCTCGTTAAGCGACTTCATTCACCT |
| pgk F | GCAAACTATGATGGGTCTTGTTGTTGGATTCTAGATAACGTGGGCGAT |
| | CGATG |
| pgk R | GGGGCTGCATCTAATAACCCTCCTTCTGATATCGCCGTACTCCTTGGA |
| | GAT |
| 21NcA F | ATCAGAAGGAGGGTTATTAGATGCAGCCCCTAGTCAG |
| NcA R | ATGCTCCTTTCCGAAACTCCGTATACTCAACGGTTTTTCAACAACCG |
| LnW F | TTCGGAAAGGAGCATCTAGGATGGATAAGATTAAACAAGGATCTGC |
| LnW R | CTTTATGCGCAACGGGATCCTTACTTTCTCCATAGCGTCACC |
| 20ABY F3 | CGTTGCGCATAAAGGAGCATCTACAATGTACTATTTAAAAAACAC |

TABLE 2
Primers

| Primer | |
|---|---|
| name | Sequence (5′→3′) |
| glmS F1 | TGCATGCCTGCAGGTCGACTTCACGAGCCCCTCATTGCCT |
| glmS R1 | CATTCGCAGGGTAACGGCCAGACTTTACAACAACTTTTTC |
| glmS F2 | GAAAAAGTTGTTGTAAAGTCTGGCCGTTACCCTGCGAATG |
| glmS R2 | ACAATTCCACACATGCGCATTGTATGTCCTCCTGGACTTC |
| glmS F3 | GAAGTCCAGGAGGACATACAATGCGCATGTGTGGAATTGT |
| glmS R3 | GCTCGGTACCCGGGGATCCTAAAGCACCCTCAAGGCGCTG |
| glmM F1 | CTATGACCATGATTACGCCACTCCGGCGAGTTCAAG |
| glmM R1 | CATTCGCAGGGTAACGGCCAGCGATTAATTATGCACGGC |
| glmM F2 | AGGCCGTGCATAATTAATCGCTGGCCGTTACCCTGC |
| glmM R2 | GTTCCAAATAGTCGAGTCATTGTATGTCCTCCTGGACTT |
| glmM F3 | GAAGTCCAGGAGGACATACAATGACTCGACTATTTGGAACTG |
| glmM R3 | TTGTAAAACGACGGCCAGTGTTCAGGTGCTCTAGGTAACGG |
| glmU F1 | TGCATGCCTGCAGGTCGACTCTCTGGAATCTGGTCGGATC |
| glmU R1 | CATTCGCAGGGTAACGGCCAGATTATCTCAAATCCTTAAA |
| glmU F2 | TTTAAGGATTTGAGATAATCTGGCCGTTACCCTGCGAATG |
| glmU R2 | GAGAAATCGCTTGCGCTCAATGTATGTCCTCCTGGACTTC |
| glmU F3 | GAAGTCCAGGAGGACATACATTGAGCGCAAGCGATTTCTC |
| glmU R3 | GCTCGGTACCCGGGGATCCTTGCTCAACGATGGCGGTGAC |
| pgm F1 | TGCATGCCTGCAGGTCGACTACACGCCAGGGTATTCGCCG |
| pgm R1 | CATTCGCAGGGTAACGGCCAGTTTGCTCCTTAAAACACCA |
| pgm F2 | TGGTGTTTTAAGGAGCAAACTGGCCGTTACCCTGCGAATG |
| pgm R2 | CCGGCGCGTTCATGTGCCATTGTATGTCCTCCTGGACTTC |
| pgm F3 | GAAGTCCAGGAGGACATACAATGGCACATGAACGCGCCGG |
| pgm R4 | GCTCGGTACCCGGGGATCCTTTGTATTTGAATCCGCCATC |
| galU1 F1 | TGCATGCCTGCAGGTCGACTTCGTAGAAACCGCCACCTTT |
| galU1 R1 | CATTCGCAGGGTAACGGCCAGGAACCAAGAGTACCTGCCC |
| galU1 F2 | GGGCAGGTACTCTTGGTTCCTGGCCGTTACCCTGCGAATG |
| galu1 R2 | TCATCGATAGGCAAACTCATTGTATGTCCTCCTGGACTTC |
| galU1 F3 | GAAGTCCAGGAGGACATACAATGAGTTTGCCTATCGATGA |
| galU1 R3 | GCTCGGTACCCGGGGATCCTCAAAGGACAGATCCACCG |
| galE F1 | TGCATGCCTGCAGGTCGACTCTCCAGAGGGACGTTCCCTC |
| galE R1 | CATTCGCAGGGTAACGGCCACGTGTGTTAGCCCTCAACCT |
| galE F2 | AGGTTGAGGGCTAACACACGTGGCCGTTACCCTGCGAATG |
| galE R2 | CCGGTAACCAGAAGCTTCATTGTATGTCCTCCTGGACTTC |
| galE F3 | GAAGTCCAGGAGGACATACAATGAAGCTTCTGGTTACCGG |
| galE R3 | GCTCGGTACCCGGGGATCCTAAGTAGCGCAAGCTGGTTGC |

TABLE 3

| Strains | Related characteristics |
|---|---|
| E. coli TOP10 | F, mrcA Δ(mrr-hsdRMS-mcrBC) |
| | φ80lacZΔM15 |
| | lacX74 recA1 araD139Δ(ara-leu) 7697 |
| | galU galK rpsL (Str^{R}) endA1 nupG |
| E. Coli K-12 MG1655 | F⁻, lambda⁻, rph-1 |
| C. glutamicum | Wild-type strain, ATCC13032 |
| C. glutamicum P | P_{tuf}-pgm |
| C. glutamicum U | P_{tuf}-galU1 |
| C. glutamicum E | P_{tuf}-galE |
| C. glutamicum PU | P_{tuf}-pgm, P_{tuf}-galU1 |
| C. glutamicum PE | P_{tuf}-pgm, P_{tuf}-galE |
| C. glutamicum VE | P_{tuf}-galU1, P_{tuf}-galE |
| C. glutamicum PUE | P_{tuf}-pgm, P_{tuf}-galUl, P_{tuf}-galE |
| C. glutamicum S | ATCC13032 P_{tuf}-glmS |
| C. glutamicum M | ATCC13032 P_{tuf}-glmM |
| C. glutamicum U | ATCC13032 P_{tuf}-glmU |
| C. glutamicum SM | ATCC13032 P_{tuf}-glmS P_{tuf}-glmM |
| C. glutamicum SU | ATCC13032 P_{tuf}-glmS P_{tuf}-glmU |
| C. glutamicum MU | ATCC13032 P_{tuf}-glmM P_{tuf}-glmU |
| C. glutamicum SMU | ATCC13032 P_{tuf}-glmS P_{tuf}-glmM Ptuf- |
| | glmU |

TABLE 4
Plasmids

| Plasmids | Related characteristics |
|---|---|
| pCN013 | Kan^{R}, pUC origin of replication, |
| | Tuf(p), T7 terminator, 6xHis |
| | affinity tag |
| pAY | pCN013 + 21RBS-lgtA-LacYA |
| pAWY | pCN013 + lgtA-WbgO-lacY |
| pABY | pCN013 + lgtA-lgtB-lacY |
| pKmobsacB | Kan^{R}, mobilizable E. coli vector for |
| | the construction of insertion and |
| | deletion mutants of C. glutamicum |
| | (oriV, sacB, lacZ) |
| pK19mobsacB-tuf-glmS | pKmobsacB + 500 base pair upstream of |
| | glmS gene-Tuf(p) glmS 500 base pair |
| pK19mobsacB-tuf-glmM | pKmobsacB + 500 base pair upstream |
| | of glmM gene-Tuf(p)-glmM 500 base pair |
| pK19mobsacB-tuf-glmU | pKmobsacB + 500 base pair upstream |
| | of glmU gene-Tuf(p)-glmU 500 base pair |
| pK19mobsacB-tuf-pgm | pKmobsacB + 500 base pair upstream |
| | of pgm gene-Tuf(p)-pgm 500 base pair |
| pK19mobsacB-tuf-GalU1 | pKmobsacB + 500 base pair upstream |
| | of GalU1 gene-Tuf(p)-GalU1 500 base pair |
| pK19mobsacB-tuf-GalE | pKmobsacB + 500 base pair upstream |
| | of GalE gene-Tuf(p)-GalE 500 base pair |

TABLE 5
Gene sequences

| | | Codon-optimized |
|---|---|---|
| | | for expression in |
| | | Corynebacterium |
| Gene name | SEQ ID NO: | glutamicum |
| lgtA (β-1, 3-N- | SEQ ID NO: 1 | X |
| acetylglucosaminyltransferase) - | | |
| Neisseria cinerea ATCC 14685 | | |
| lgtA (β-1, 3-N- | SEQ ID NO: 2 | X |
| acetylglucosaminyltransferase) - | | |
| Neisseria meningitidis M98 | | |
| lgtB (β-1,4- | SEQ ID NO: 3 | X |
| galactosyltransferase) - | | |
| Neisseria cinerea ATCC 14685 | | |
| WbgO (β-1, 3- | SEQ ID NO: 4 | X |
| galactosyltransferase) - | | |
| Lutiella nitroferrum | | |
| ATCC BAA-1479 | | |
| lacY (lactose permease) | SEQ ID NO: 5 | X |

### Example 2: Culture Conditions and Method of Recombinant Corynebacterium glutamicum

For seed culture, a glass test tube containing 4 mL BHI (brain heart infusion) medium supplemented with appropriate antibiotics (kanamycin 25 μg/mL) was used, and the culture was performed at a stirring rate of 250 rpm for 12 hours while maintaining the temperature at 30° C.

The culture was performed in a flask culture using 40 mL of CGXII (5 g/L of urea, 0.25 g/L of MgSO₄, 42 g/L of MOPS, 1 g/L of potassium phosphate monobasic, 1 g/L of potassium phosphate dibasic, 10 mg/L of CaCl₂, 0.2 mg/L of biotin, 30 mg/L of protocatechuic acid, 10 mg/L of FeSO₄7H₂O, 10 mg/L of MnSO₄H₂O, 1 mg/L of ZnSO₄7H₂O, 0.2 mg/L of CuSO₄, 0.02 mg/L of NiCl₂6H₂O, glucose of 20 g/L, 5 g/L of lactose, pH 7.0) medium supplemented with appropriate antibiotics (kanamycin 25 μg/mL) at a temperature of 25° C. and a stirring rate of 200 rpm for 72 hours.

### Experimental Example 1: Determination of Concentration of Cells and Metabolites and Comparison of Productivity

1) Experimental Method for Determination of Concentration of Cells and Metabolites and Comparison of Productivity

To compare the productivity of LNT, LNnT, and LNTII, culture was performed using a glass test tube containing 4 L BHI (brain heart infusion) medium supplemented with antibiotics (25 μg/mL of kanamycin) at a temperature of 30° C. and a stirring rate of 250 rpm for 12 hours, the medium was inoculated into a shaking flask containing 40 mL CGXII medium supplemented with 25 μg/mL of kanamycin to an initial O.D. (optical density) of 0.3. Culture was performed in the medium at a culture temperature of 25° C. and a stirring rate of 200 rpm for 72 hours. After culturing for 72 hours, 1 ml of the culture solution was dispensed into a 1.7 ml tube and boiled at 95° C. The boiled culture medium was centrifuged at 15,000 rpm for 1 minute, and the supernatant was diluted 100-fold and analyzed for concentration using HPLC. The concentrations of LNT, LNnT, LNTII, lactose, lactate, glucose, and acetic acid were measured using HPLC (high performance liquid chromatography) (Agilent 1260, USA) equipped with a carbohydrate analysis column (Aminex HPX87H column, Bio-rad) and an RI (refractive index) detector. 20 μl of culture medium was analyzed using a column heated at 60° C. 5 mM H₂SO₄ solution was used as a mobile phase at a flow rate of 0.6 mL/min.
2) Comparison of LNT II Productivity

The strain of Example 1, which was produced to overexpress glms, glmM, and glmU of the production pathway of UDP-N-acetylglucosamine, a precursor for LNT/LNnT production, was used to compare the production of LNTII, a precursor of LNT/LNnT, using the productivity comparison experiment method described above.

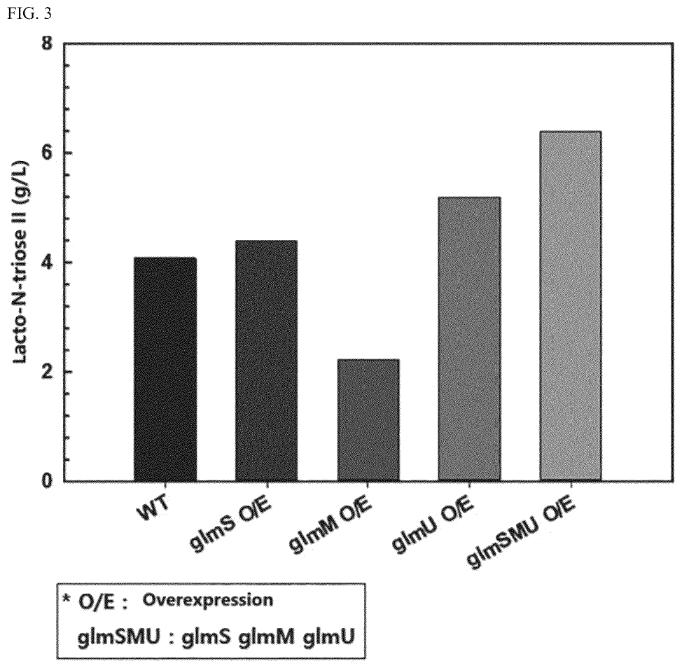

The result showed that the production of LNTII was remarkably increased in glmSMU O/E, which overexpressed glmS, glmM, and glmU of the UDP-N-acetylglucosamine production pathway, as shown in FIG. 3.
3) Comparison in Productivity between LNT and LNnT

The strain of Example 1, which was produced to overexpress pgm, galU, and galE of the production pathway of UDP-galactose, a precursor for LNT/LNnT production, was used to compare the production of LNT/LNnT using the productivity comparison experiment method (PU O/E: pgm GalU O/E; PE O/E: pgm GalE O/E; UE O/E: GalU GalE O/E; PUE O/E: pgm GalU GalE O/E).

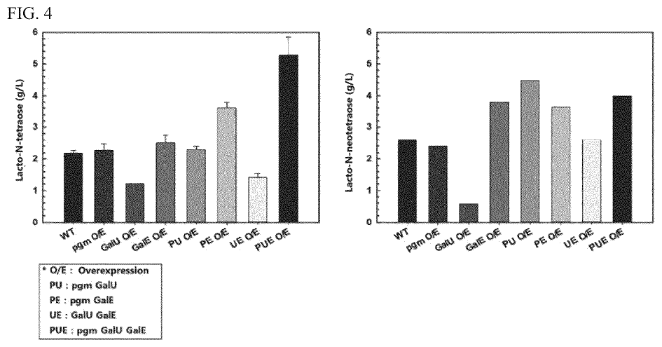

As a result, as shown in FIG. 4, the production (final production) of LNT increased the most when pgm, galU, and galE were all overexpressed (PUE O/E), and that the production (final production) of LNnT increased the most when pgm and galU were overexpressed (PU O/E) than when pgm, galU, and galE were all overexpressed (PUE O/E).

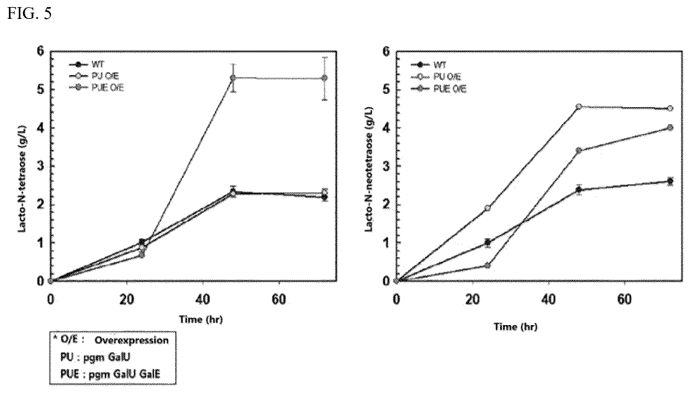

Meanwhile, regarding the changes in the production of LNT and LNnT over time, as shown in FIG. 5, the production of LNT increased the most when pgm, galU, and galE were all overexpressed (PUE O/E), and the production of LNnT increased the most when pgm and galU were overexpressed (PU O/E) compared to when pgm, galU, and gale were all overexpressed (PUE O/E).

1. Recombinant Corynebacterium glutamicum transformed such that exogenous genes, including genes encoding lactose permease, genes encoding β-1, 3-N-acetylglucosaminyltransferase, and genes encoding (62 -1, 3-galactosyltransferase are expressed in Corynebacterium glutamicum,
the recombinant Corynebacterium glutamicum transformed such that one or more genes selected from endogenous genes in Corynebacterium glutamicum, including genes encoding glutamine-fructose-6-phosphate aminotransferase, genes encoding phosphoglucosamine mutase, genes encoding glucosamine-1-phosphate N-acetyltransferase, genes encoding UDP-N-acetylglucosamine pyrophosphorylase, genes encoding phosphoglucomutase, genes encoding UTP-glucose-1-phosphate uridylyltransferase, and genes encoding UDP-glucose-4-epimerase are overexpressed.

2. Recombinant Corynebacterium glutamicum transformed such that exogenous genes, including genes encoding lactose permease, genes encoding β-1, 3-N-acetylglucosaminyltransferase, and genes encoding β-1, 4-galactosyltransferase, are expressed in Corynebacterium glutamicum,
the recombinant Corynebacterium glutamicum transformed such that one or more genes selected from endogenous genes in Corynebacterium glutamicum, including genes encoding glutamine-fructose-6-phosphate aminotransferase, genes encoding phosphoglucosamine mutase, genes encoding glucosamine-1-phosphate N-acetyltransferase, genes encoding UDP-N-acetylglucosamine pyrophosphorylase, genes encoding phosphoglucomutase, genes encoding UTP-glucose-1-phosphate uridylyltransferase, and genes encoding UDP-glucose-4-epimerase, are overexpressed.

3. A method for producing lacto-N-tetraose comprising culturing the recombinant Corynebacterium glutamicum according to claim 1 in a medium containing lactose.

4. The method according to claim 3, wherein the medium further contains glucose.

5. A method for producing lacto-N-neotetraose comprising culturing the recombinant Corynebacterium glutamicum according to claim 2 in a medium containing lactose.

6. The method according to claim 5, wherein the medium further contains glucose.
